(19) United States
(12) Patent Application Publication
(10) Pub. No.: US 2024/0347212 A1
(43) Pub. Date: Oct. 17, 2024

(54) METHODS AND SYSTEMS FOR PERFORMING CONTACT TRACING

(71) Applicant: Intelligent Locations, Inc., Raleigh, NC (US)

(72) Inventors: Bogdan Nedelcu, Wake Forest, NC (US); Charles Dias, Paris (FR)

(73) Assignee: Intelligent Locations, Inc., Raleigh, NC (US)

(21) Appl. No.: 18/196,948

(22) Filed: May 12, 2023

Related U.S. Application Data
(60) Provisional application No. 63/341,760, filed on May 13, 2022.

(51) Int. Cl. G16H 50/80; H04W 4/021

(52) U.S. Cl. CPC G16H 50/80; H04W 4/021

(57) ABSTRACT

Real time localization systems are provided that have the ability to locate people in an area. By leveraging signal data gathered by these systems, methods for enabling precise contact tracing in time and space are provided. These systems and methods enable applications such as automatic contact tracing and the automatic computation of risk reduction through attendance reduction.

### RELATED APPLICATIONS

This application claims the benefit of and priority to U.S. Provisional Application No. 63/341,760, filed May 13, 2022, the contents which is hereby incorporated herein by reference in its entirety.

### FIELD

The present disclosure uses radio technology to determine the location of the assets but also to determine their status for the purposes of contact tracing.

### BACKGROUND

To avoid epidemic spread of airborne diseases, it is essential to test and quarantine ill persons. However, finding healthy people who were in contact with infected individuals can be complicated. This is generally done via “contact tracing.” Contact tracing usually relies on a thorough interview process where the infected person is asked where he was and who he saw. This process is long, humanly expensive and error prone. It can be important to perform contact tracing with a more reliable and more error-proof process.

### SUMMARY

The present disclosure is directed to systems and methods for contact tracing.

A contact tracing system is provided and can include a plurality of aggregators configured to communicate in a meshed network to create a geo-fence within a designated area, each of the plurality of aggregators including a processor and a transceiver, and one or more sensors, each of the one or more sensors associated with an asset, the one or more sensors configured to communicate, with the plurality of aggregators, information relating to a location of the asset based on a relative location of the asset to each of the plurality of aggregators. The system includes a central computer in communication with the plurality of aggregators and configured to receive information from at least one of the plurality of aggregators relating to a contact tracing event within the geo-fence within the designated area, the central computer configured to receive, from at least one of the plurality of aggregators, a plurality of parameters relating to an asset in the designated space; and determining a contact status of the asset based on the plurality of parameters, the plurality of parameters including a duration of time the at least one asset remained in the designated area and a distance between the at least one asset and one or more additional assets in the designated area.

In some embodiments, the central computer is configured to calculate a proximity score using one or more signals from the sensors as captured by each aggregator, the one or more signal being transformed into a single numerical vector, the proximity score being a similarity between a first numerical vector related to the at least one asset and a second numerical vector related to one or the one or more additional assets. In some embodiments, the central computer is configured to compare the proximity score with a stored proximity threshold value to determine if the asset and the additional asset are in close proximity indicative of spreading disease.

In some embodiments, the central computer is configured to calculate a contact duration score using timestamps of all the signals received by the aggregators in the designated area. In some embodiments, the central computer is configured to compare the contact duration score with a stored duration threshold value to determine if the asset and the additional asset are in contact long enough to be indicative of spreading disease.

In some embodiments, the central computer is configured to create a contact database of contacts between the asset and the one or more additional assets for automatic contact tracing.

A method of contact tracing can be provided and can include transmitting a plurality of parameters from one or more of a plurality of aggregators relating to an asset to a central computer, the asset including one or more tags positioned thereon for communicating with the plurality of aggregators in a designated area, determining a localization, by the central computer, of the asset in the designated area based on the plurality of parameters, determining a contact between the asset and one or more additional assets based on the determined localization of the asset and the one or more additional assets, determining a contact status of the asset based on the plurality of parameters using an algorithm on the central computer, the plurality of parameters including a duration of time the asset remains in the designated area and a distance between the asset and one or more additional assets in the designated area, and determining a close contact between the asset and the one or more additional assets, a close contact defined as a contact between the asset and the additional asset indicative of spreading disease.

In some embodiments, the method can further include calculating a proximity score using one or more signals from the tags as captured by each aggregator, the one or more signals being transformed into a single numerical vector, the proximity score being a similarity between a first numerical vector related to the asset and a second numerical vector related to one or the one or more additional assets. In some embodiments, the method can further include comparing the proximity score with a stored proximity threshold value to determine if the asset and the additional asset are in close proximity indicative of spreading disease.

In some embodiments, the method can further include calculating a contact duration score using timestamps of all the signals received by the aggregators in the designated area. In some embodiments, the method can further include comparing the contact duration score with a stored duration threshold value to determine if the asset and the additional asset are in contact long enough to be indicative of spreading disease.

In some embodiments, the method can further include generating an alert if the contact is determined to be a close contact indicative of spreading disease.

In some embodiments, the central computer creates a contact database of the close contacts between the asset and the one or more additional assets for automatic contact tracing.

In some embodiments, the method can further include creating a geo-fence within the designated area defining the designated area using a plurality of aggregators, each aggregator capable of communicating with neighboring aggregators and with the central computer over a network.

A contact tracing method can be provided and can include transmitting a plurality of parameters from one or more of a plurality of aggregators relating to one or more assets to a central computer, each of the one or more assets including one or more tags positioned thereon for communicating with the plurality of aggregators in a designated area, the one or more assets including at least a first asset and a second asset, determining, by a central computer, a proximity score related to a proximity between the first asset and the second asset based on the plurality of parameters from the plurality of parameters, determining, by a central computer, a duration score related to a duration of time the first asset and the second asset are in the designated area based on the plurality of parameters from the plurality of parameters, and determining a contact status of the first asset and the second asset based on the proximity score and the duration score using an algorithm on the central computer. The algorithm can compare the proximity score to a threshold proximity value stored on the central computer and comparing the duration score to a threshold duration value stored on the central computer.

In some embodiments, the contact status is determined to be a close contact when the proximity score exceeds the threshold proximity value and the duration score exceeds the threshold duration value, indicating a contact that is indicative of spreading disease. In some embodiments, the central computer creates a contact database of the close contacts between the asset and the one or more additional assets for automatic contact tracing.

In some embodiments, the method can further include creating a geo-fence within the designated area defining the designated area using a plurality of aggregators, each aggregator capable of communicating with neighboring aggregators and with the central computer over a network.

In some embodiments, the method can further include calculating a proximity score using one or more signals from the tags as captured by each aggregator, the one or more signal being transformed into a single numerical vector, the proximity score being a similarity between a first numerical vector related to the at least one asset and a second numerical vector related to one or the one or more additional assets.

In some embodiments, the method can further include calculating a contact duration score using timestamps of all the signals received by the aggregators in the designated area.

In some embodiments, a method is provided that includes transmitting parameter data relating to an asset, such as a person, to a central computer, the asset includes one or more tags positioned thereon for transmitting the parameter data and determining a status of the asset based on the parameter data using an algorithm on the central computer. This information is used to perform contact tracing and risk assessment.

In some embodiment, the method further includes creating a geo-fence within a space defining the specified area using a plurality of aggregators, with each aggregator capable of communicating with neighboring aggregators and with the central computer over a network. The method includes determining a location of the asset within the area of the geo-fence and determining the location and/or duration in the location of the asset relative to the aggregators forming the geo-fence.

In some embodiments, a method is provided that includes selecting an asset from a list of assets on a mobile application located in a specified area, transmitting the selected asset information from the mobile application to a central computer instructing the central computer to increase the transmission rate of information from one or more tags positioned on the asset, the one or more tags positioned thereon for transmitting a parameter data related to the location of the asset, transmitting a message comprising the increased transmission rate to the tag of the asset, and determining the location of the asset based on the parameter data transmitted from the tag at the increased transmission rate using an algorithm on the central computer. The transmission rate is reset to an initial transmission rate when the asset has been located, the initial transmission rate being lower than the increased transmission rate.

In some embodiments, a method is provided that includes transmitting parameter data relating to an asset to a central computer, the asset includes one or more tags positioned thereon for transmitting the parameter data and determining a status of the asset based on the parameter data using an algorithm on the central computer. The status of the asset is used to compile a profile of the asset over time to determine a usage rate of the asset.

### BRIEF DESCRIPTION OF THE DRAWINGS

The present disclosure is further described in the detailed description which follows, in reference to the noted plurality of drawings by way of non-limiting examples of exemplary embodiments, in which like reference numerals represent similar parts throughout the several views of the drawings, and wherein:

FIG. 1 illustrates an exemplary flowchart of a localization algorithm;

FIG. 2 is an embodiment of a system for determining the location of an asset:

FIG. 3 illustrates an exemplary flowchart of a process of building a database of contacts:

FIG. 4 illustrates an exemplary flowchart of a process for computing duration and proximity:

FIG. 5A and FIG. 5B illustrate exemplary graphs showing a first refinement in the translation of abstract raw proximity scores to a value in meters:

FIG. 6 illustrates an exemplary embodiment of a graph of time versus proximity to show a computed real contact time using a safe distance threshold:

FIG. 7 illustrates an exemplary flowchart of a process of determining potentially disease-spreading encounters:

FIG. 8 illustrates an exemplary embodiment of a graph showing risk score for categorizing each susceptible person:

FIG. 9 illustrates an exemplary flowchart of a contact alert procedure:

FIG. 10 illustrates an exemplary embodiment of using a forecasting method/algorithm along with risk contact parameters:

FIG. 11 illustrates an exemplary embodiment of a flowchart showing a method of using low attendance to adjust risk assessment:

FIG. 12 illustrates an exemplary 3D view of the displacement of an asset:

FIG. 13 is an embodiment of a system for determining a location and/or status of an asset:

FIG. 14 illustrates an embodiment of a user interface of a mobile application for locating an asset:

FIG. 15 illustrates an embodiment of a flowchart for determining the location of an asset using a change in a transmission rate of signals from the asset.

FIG. 16 illustrates an embodiment of a system that is configured to determine a location and/or status of an asset:

FIG. 17 illustrates an exemplary embodiment of a client device; and

FIG. 18 illustrates an embodiment of an internal architecture of a computing device, computing system, and computing platform.

### DETAILED DESCRIPTION

The present disclosure describes systems and methods to track movement of an asset, such as personnel, through an area in order to perform contact tracing as it relates to airborne illnesses or other diseases using, for example, a meshed network or similar network with radio coverage within a designated area, such as a building, hospital, or warehouse.

In some embodiments, system and methods are provided to automatically accomplish contact tracing in a closed facility, such as a hospital, using real time indoor localization. By leveraging the localization and radio signal strength, the system can trace, score and rank contacts between people. This system can directly help decision-makers limit the spread of the disease inside their establishment. A direct application is the control of Hospital Acquired Infections (HAI), including various airborne illness like COVID-19.

In some embodiments, a contact tracing system can include contact logging, computation of contact parameters, automatic risk assessment, visualization and alerts. A contact can be defined as two people at a specific distance (d) for a certain amount of time (t). The system can use various algorithms to define when a contact becomes high risk, for example, if a close contact lasts a certain amount of time. Therefore, the contact “risk” can be defined as an aggregate of its distance and its time.

In some embodiments, an algorithm provides the location of an asset, such as a person, and can perform a risk analysis related to contact tracing for an airborne illness based on inputs automatically generated relating to location and time spent in a location relative to another asset using one or more sensors/tags attached thereto. The sensors are active and can broadcast an identifier and/or payload data such as 3D coordinates and duration information. This data is sent to aggregators that are fixed and located anywhere in an area, such as a hospital, to achieve ubiquitous (or approximately ubiquitous or not ubiquitous) coverage so that they can capture the sensor's signals (and data). The aggregators transmit the data received from sensors and send it to a central computing system where the algorithm resides. The algorithm uses the data to indicate risk related to contact tracing information.

Location information can be used to determine if more than one person is in a certain area at the same time. Accelerometer information can be used to track movement of the people in an area relative to one another. Duration information can be used to track the amount of time two or more people are located in the same area as one another, and this can used in conjunction with location and proximity information to determine if a contact has occurred and access the risk of that contact.

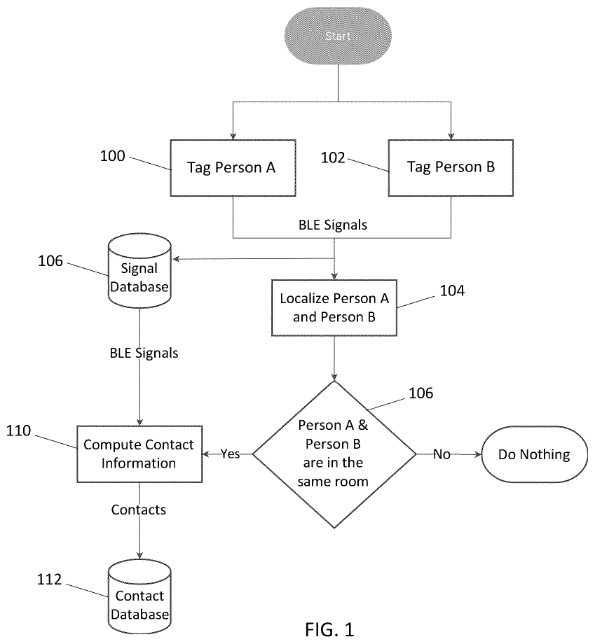

In some embodiments, a localization algorithm enables the system to determine when two or more people are in the same room. However, two people in the same room can be so far apart that they are not really in contact. As shown in FIG. 1, the system can augment the localization algorithm to tackle this issue. In some embodiments, a contact between two people can be refined using the Bluetooth signals (for example Bluetooth low energy, or BLE, signals), precisely timed and augmented with a proximity measure. This enables more precise contact tracing. Specifically, it becomes possible to sort contact by risk and produce detailed contact tracing reports. Contacts are by definition two people at a specific distance (d) for a certain amount of time (t). By definition a contact becomes risky when it's a close contact that lasts a certain amount of time. Therefore, the contact “risk” can be defined as an aggregate of its distance and its time.

It can be understood that the area (for example, a hospital, a warehouse, etc.) is covered by a network of aggregators placed such that they provide coverage (ubiquitous or not) in the areas of interest of the hospital/warehouse. In some embodiments, the aggregators can be in communication with a computing device, such as a central computer, via a network, such as the Cloud. The central computer has a computing function and a database repository for all data needed to be stored or data which is recorded from various operations, including but not limited to events, measurements, etc.

The systems and methods described herein can be used in a variety of applications where there is a need to remotely perform contact tracing of a plurality of assets, including but not limited to the medical field. A combination of parameters, such as data relating to duration and distance between two or more people, can provide a more idea of the status of an asset (i.e., a person). It will be understood that information regarding more than one parameter increases the precision of information regarding the related asset.

To do contact tracing, the system is configured to employ person tracking features. In some embodiments, BLE tags are given to people (i.e., patients, doctors, other staff, and/or anyone who enters a specified area) and Bluetooth gateways are installed in facilities. For example, every tracked person has a tag, and every tag sends Bluetooth signals to the installed gateways. In some embodiment, the BLE signals are stored in a signal database 106. For example, as shown in FIG. 1, person A and person B can be given a tag (steps 100, 102) The sent Bluetooth packets are redirected to a cloud platform which computes localization (step 104) and stores its history. This setting enables localization and tracking of individuals within equipped facilities. The localization setting can be used to find, score and log contacts between people. For example, the system can decide if there is a contact between person A and person B, such as that they are in the same room (step 108). Leveraging these logged contacts, the system can automatically assess whether or not a contact between two individuals (i.e., patients of an hospital) is at risk of transmitting an airborne disease by computing contact information (step 110). In some embodiment, the contact information can be stored, for example, in a contact database 112.

The one or more sensors are attached to the assets, such as people in the area. The sensor can be in the form of Bluetooth or other wireless technologies embedded in a tag. The tag is a small circuit board, powered by a small battery which can send radio signals at regular intervals and with a certain power. The parameters (for example, signal frequency and power levels) are adjustable in software. The signals can be of different radio technologies (Bluetooth, Zigbee, Wi-Fi, Ultra-Wide Band, etc.) and contain any type of payload data, or no payload at all. Since they are independently powered, they can be appended to any asset and move with the asset. The fixed aggregators will receive the message sent by the tags, send them to the cloud, or other network, where an algorithm running on the central computer decodes the values and makes a determination as to the action/no action needed to be taken to update the status of the asset (for example, relating to contact between two or more people in the area).

In some embodiments, a geo-fence can be created in an area using the plurality of aggregators. In some embodiments, the geo-fence and aggregators can be used to determine when a person enters or leaves the area. For example, if an asset breaks the geo-fence created by the wireless network formed by the aggregators and/or the temperature changes suddenly, it is very likely that the asset is outside the area. The central computer will record the last know aggregator which received the signals from the tag appended to the asset and the time. This can allow the system to log when a person leaves the area covered by the aggregators, for example a hospital setting.

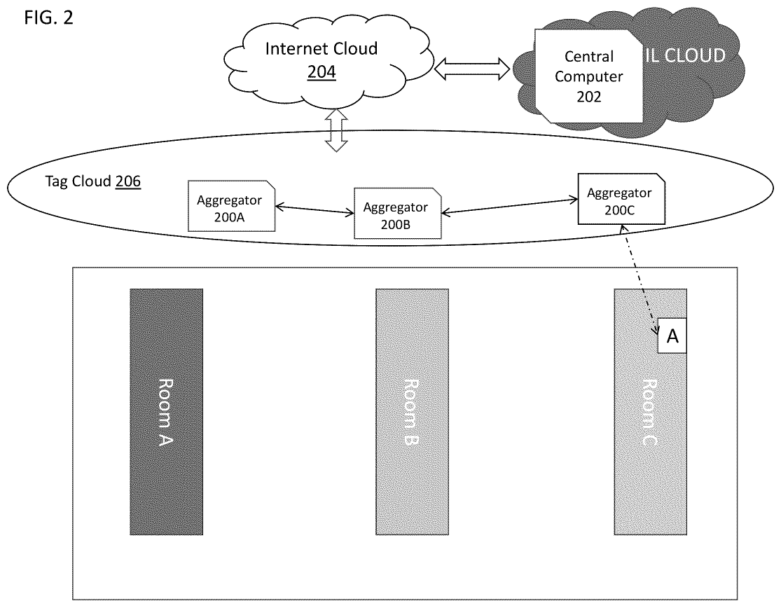

FIG. 2 illustrates an embodiment of components of the system showing the location of an asset “A” (i.e., person A) determined by an aggregator, for example an aggregator 200C. As shown in FIG. 2, there are a plurality of aggregators 200A, 200B, 200C creating a tag cloud 206 in communication with a central computer 202 using a cloud 204.

In some embodiments, spatial displacement of the tag relative to the aggregators can determine location of the one or more tags relative to one another in an area. The aggregators can forward the data to the status determination algorithms in a backend server (or the central computer). The backend server will analyse the location data of the tag within the geo-fence formed by the aggregators. If the analysis yields a determination that the tags have met parameters relating to a close contact, the status of the each of those tags can be set to “close contact.”

### Finding and Scoring Contacts

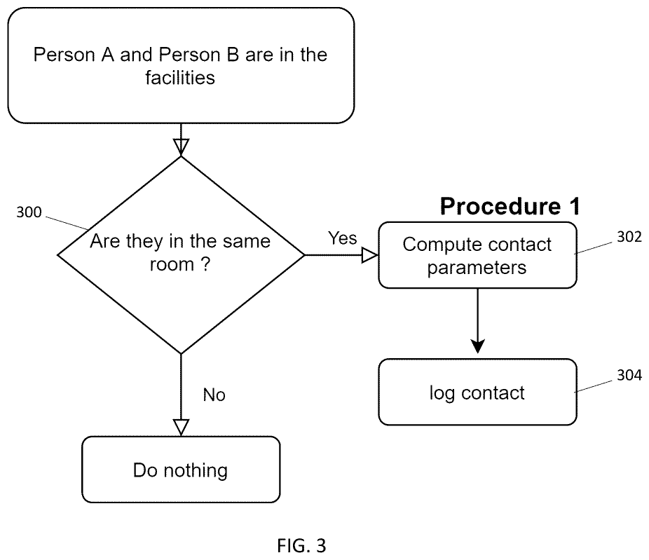

Leveraging a real time localization system (RTLS) and all the Bluetooth signal information it collects, the system can process and store all contacts happening between individuals within an area or a facility equipped with the localization system. The process of building a database of contacts is shown in FIG. 3. Given two people, for example Person A and Person B, the system can determine if person A and Person B are in the same area (step 300), for example the same room, compute contact parameters (step 302) and log a contact (step 304) if Person A and Person B are both in the same area, such as the same room, at the same time. One or more parameters, for example, extracted from the Bluetooth signal data, are also associated with each contact, including but not limited to its localization, duration, and a proximity between the two people. Whenever two Persons A and B are in the same room or in the same area, a computation of contact parameters can be performed (Procedure 1, described below). Finally, the contact along with its parameters are stored in a database for further use. Given a list of people in a same room at the same time, this process is generalized to every unique pair. This makes it possible to log contacts of a large number of people within the same area. Running this process in real-time enables us to find and log every contact in equipped facilities.

### Computing Contacts and Contact Parameters

For airborne diseases transmission, in addition to contact information relating to location and persons, at least two parameters are relevant: the duration and the proximity of the contact. The infection transmission probability is highly correlated with those two parameters.

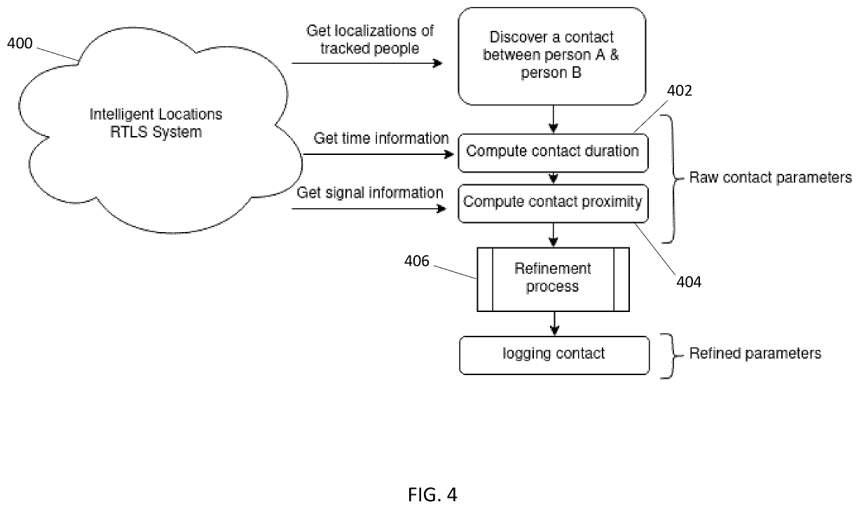

FIG. 4 illustrates an exemplary embodiment of an RTLS 400 for computing duration and proximity. As described above, the localization system enables the system to discover contacts between two people. When one contact is found, raw duration 402 and raw proximity 404 are computed (in addition to contact information).

In some embodiments, raw proximity is computed using the RSSI BLE signal information from the RTLS system. Each person signals' are captured by every aggregator and are joined within one single numerical vector. Hence one person is represented by the sum of its signals. Then a raw proximity score is computed as a normalized scalar product between the single numerical vector from person A and the single numerical vector from person B. It quantifies the similarity between the two distributions of signals. The normalization takes into account multiple parameters such as the number of gateways receiving a signal and each dimension of these numerical vectors represent one aggregator. In some embodiments, this parameter is a score bounded between 0 and 1. A score of 0 means that the two signals are dissimilar (i.e., it was a far contact) and a score of 1 means that the two signals are similar (i.e., it was a close contact). Raw contact duration is computed using the RTLS timestamps extracted from the system. It is computed as the time that person A and person B spent in the same room. For example, for one localization l (person p, room r, time t), the RTLS timestamp is extracted from the minimum timestamp of all the signals received by the aggregators of the room r. For example, for one contact between two person A and B in the same room R, there can be four location timestamps (A1, A2, B1, B2) stemming from the RTLS system: (A1) A enters room R. (B1) B enters room R. (A2) A exits room R, and (B2) B exits room R. Concretely, the raw contact duration is the time between MAX (A1,B1) and MIN (A2,B2).

Afterwards, once these raw parameters have been computed, the raw parameters can be used as inputs a refinement process/algorithm 406. It can refine the parameters in two ways: 1) the proximity score can be translated into approximate meters, and 2) the contact duration can be refined to use proximity information. Using the raw RTLS system information, a contact is defined as “two persons in the same room,” no matter the distance between these two people. A distance threshold is defined to categorize a contact. We can use it refine the contact duration. Concretely, the contact duration would be computed as the time spent below the distance threshold instead of the time spent in the same room. Finally, contacts along with the refined parameters are logged within a database for further use.

### Refinement Process

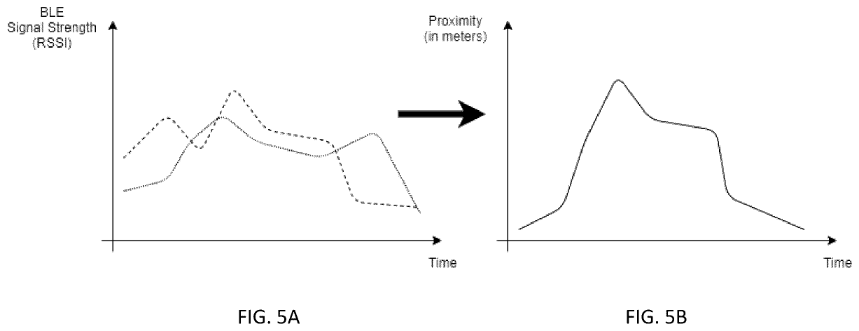

Measurements for the real time localization system need to be further processed to do contact tracing. Therefore, the automatic contact tracing method includes a refinement step which can utilize an algorithm to enable tracing contacts with higher accuracy. This process refines the contact proximity and duration parameters in two ways. The first refinement is the translation of the abstract raw proximity score to a value in meters, as shown in FIG. 5A and FIG. 5B. Indeed, the raw proximity score is a normalized similarity metric between signals it is a number between 0 and 1. Even though this value has a fixed scale, the refinement algorithm can convert this data into a more useful form in terms of contact between two or more people. In the refinement process, the raw proximity score can be translated into approximate meters. This can be formatted as a regression problem and the system can use machine learning to predict the distance between two people. Indeed, the beacons used to track people are standardized and a regressive model can be trained to achieve such a prediction. Concretely, given a dataset of couples (proximity score, distance), we can optimize a regressive model to predict the distance given the score. This predictor can also take into account side information such as the signal used to produce the proximity score. A dataset as such, of the signal data, proximity scores and distances are directly produced manually in live environments.

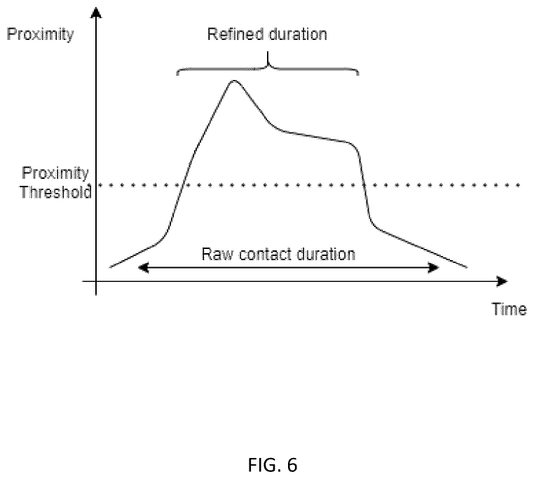

The contact duration is refined to use proximity information. The formatted data from the RTLS system only outputs when and for how long two people are in the same room, not their distance (which we compute as described above). In the refinement process, the system can use a “safe” distance threshold (for example, in meters or other measurement) to compute real contact time since a room can be sizable enough for two people to safely distance within (i.e., a large waiting room), as shown in FIG. 6.

### Leveraging the Contact Database for Contact Tracing

As explained above, RTLS can be leveraged to log all contacts happening within the equipped premises. Here, we describe how to use the created contact database for automatic contact tracing.

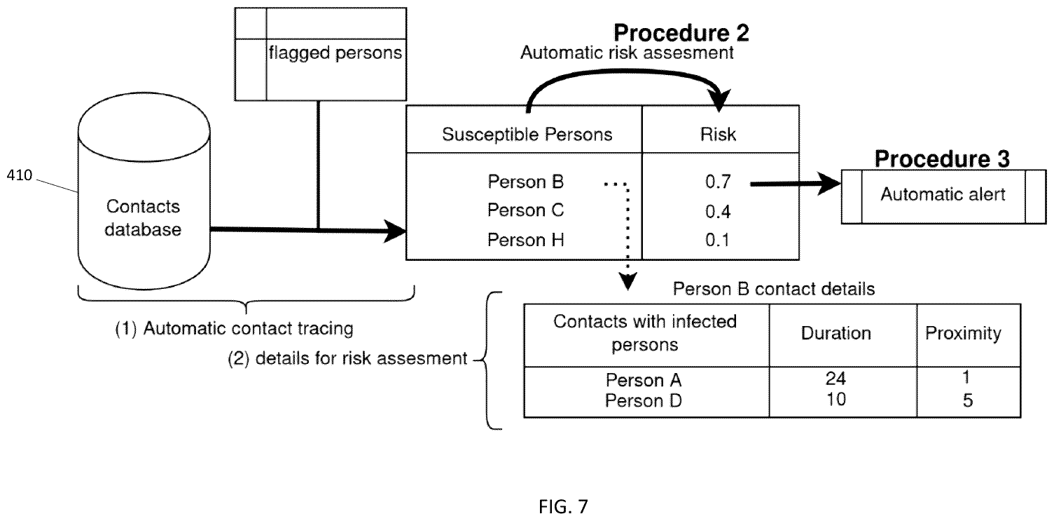

Surfacing potentially “disease-spreading encounters” is the heart of contact tracing. As depicted in FIG. 7, to do so automatically, a database 410 can maintain a record of “flagged persons” which enables the system to find susceptible persons, who are people that have had “disease-spreading encounters.” Thus, the system is enabled to find susceptible persons who have had a closed contact with an infected person in order to perform contact tracing. The method described herein can both:
(1) Find contacts between infected and healthy people. Since every contact is logged, it is possible to find who has been in contact with anyone. Thus, it is possible to automatically surface people who were in contact with any subset of “flagged” persons (i.e., infected patients). To help prioritize the search of potential susceptible contacts, the system can automatically compute a risk score—via procedure 2—of each person who had risky encounters.
(2) Assess whether or not a contact between two individuals (i.e., patients of an hospital) is at risk of transmitting any airborne disease. The refined parameters enable the system to have accurate and detailed information about the duration and the proximity of every contact. As the probability of transmission of airborne diseases greatly varies depending on duration and proximity of the contact, the detailed information enables better filtering. Based on those refined parameters, an alert system can be built that automatically triggers whenever defined thresholds are exceeded (Procedure 3).

In the following, we describe how we do automatic risk assessment (Procedure 2) and how automatic alert works (Procedure 3).

### Procedure 2: Assessing Contact Risk

The probability of transmission of airborne diseases greatly varies depending on duration and proximity of contacts. In certain settings, multiple at-risk contacts can happen. Aggregating contacts per person and sorting them per probability of infection can be used to help reveal the potential newly infected so they can be tested and eventually quarantined.

To rank each susceptible person, a “relative risk score” can be computed. In some embodiments, the relative risk score can be bounded between 0 and 1. Technically the score is a normalized combination of each person's aggregated contact time and proximity score. This combination is parametrized with an alpha (α) parameter (bounded between 0 and one) which weights the importance of proximity and time of contacts regarding the risk of transmission. The weighting is done with the following scheme: α proximity+(1−α).duration. This alpha parameter is either manually tuned by users or automatically tuned through a subsequent optimization process. The higher is alpha, the more importance we give to the proximity score with respect to the time of contacts. For example, setting alpha to 0.75 means that the final risk score would be composed of 75% of the proximity score and 25% of the duration. Concretely this would mean that the proximity of the contact is three times more important than its duration. This percentage can be either be manually tuned to reflect the user preferences. For example, the CDC Guidelines indicated that closeness of contact was less important than the time spend in the same room. If data about contact contamination is to be available, the alpha parameter can be chosen so the contacts ranked by relative risk score maximize the real contamination ranking.

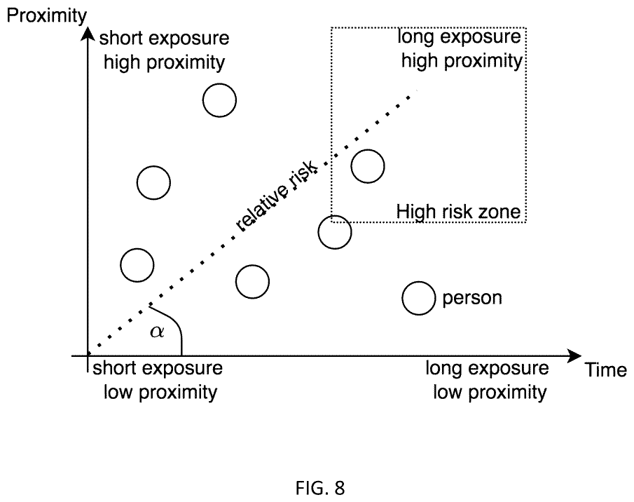

As depicted in the FIG. 8, the risk score enables the system to categorize each susceptible person. Indeed, on the 2D plane where the axis respectively represents time and proximity, four different risk zones co-exist. The upper right corner—which is where persons who had both long exposure and high proximity to infected persons are—is where the high risk individuals are. The alpha parameter enables to move the “high risk zone” towards proximity or time. Indeed, lowering alpha puts more emphasis on long contact whereas higher alpha values makes close contacts more risky.

### Procedure 3: Alert Procedure

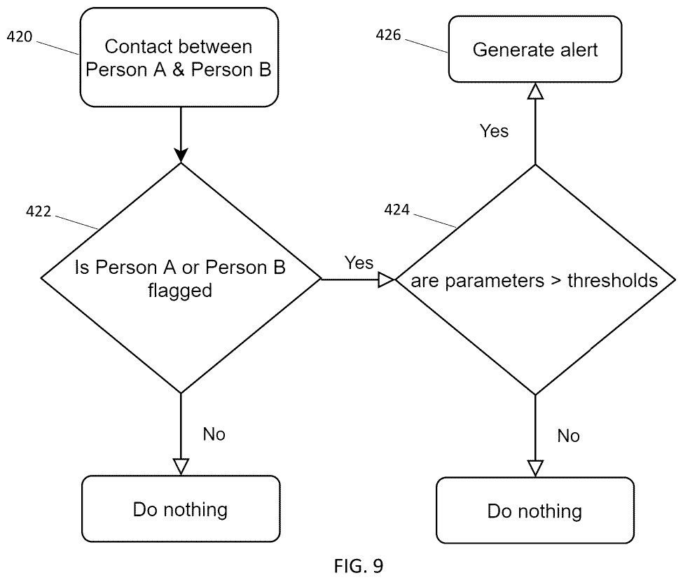

In case of an airborne disease epidemic, finding infected individuals quickly to isolate and treat them is crucial. In that sense, a contact alert procedure can be used which can work for any airborne disease (i.e., COVID-19), as shown in FIG. 9. In some embodiments, threshold parameters can be set manually. This threshold can automatically be raised to avoid false positives or lowered to avoid missing contaminative contacts. This can be done by using real testing data and optimize the threshold for maximizing contaminative contacts while minimizing false positive. Whenever a new contact is added to the contact database (step 420), if either person is flagged (step 422) and if its parameters exceed a defined threshold (step 424), an automatic alert is triggered (step 426). This enables the system to reduce manual human monitoring and makes finding contact persons to test both easy and quick.

### Leveraging the Contact Database for Social Distancing

In addition to finding potentially infected individuals through contact tracing, the system can leverage contact information to help practice social distancing. By using an automatic contact forecasting method, based on the history of contacts between individuals, a tool can predict the risk reduction (the diminution of contacts) associated with capacity reduction. Indeed, contacts are directly linked to attendance: a large facility with 1000 people would generate more contacts than the same with only 100 people. However, the reduction in attendance is surely not linearly correlated with contacts. This tool enables the system to shed light on how reducing the capacity of facilities reduces the contact risk. For example, it enables a user to see how capacity is correlated to risky contacts, with the explicit purpose of finding the best trade-off of facility usage versus risk. Choosing the best trade-off is left to the user.

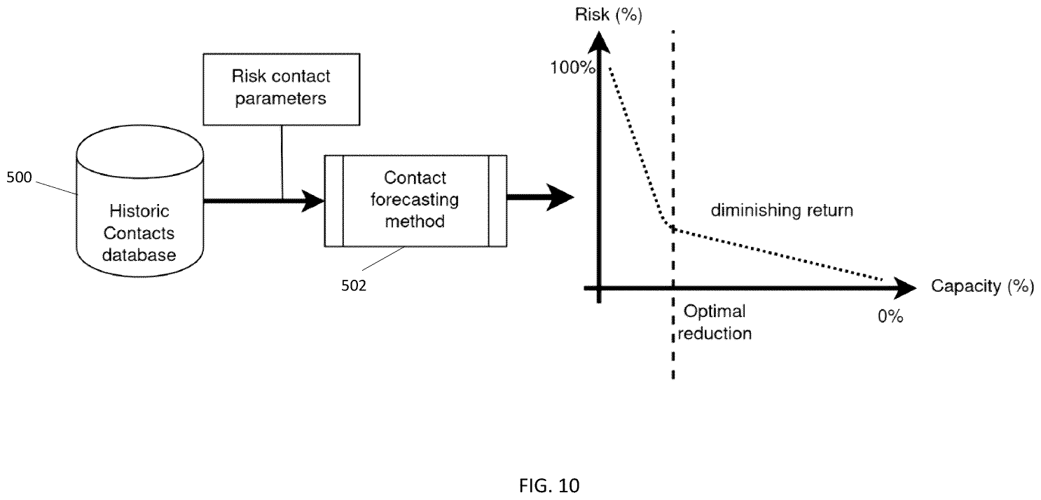

As depicted in FIG. 10, the history of contacts, for example from a contact history database 500 is directly fed to a forecasting method/algorithm 502 along with risk contact parameters. This enables the system to compute the link between capacity and risk. Leveraging this information, it becomes possible to optimize the capacity of facilities.

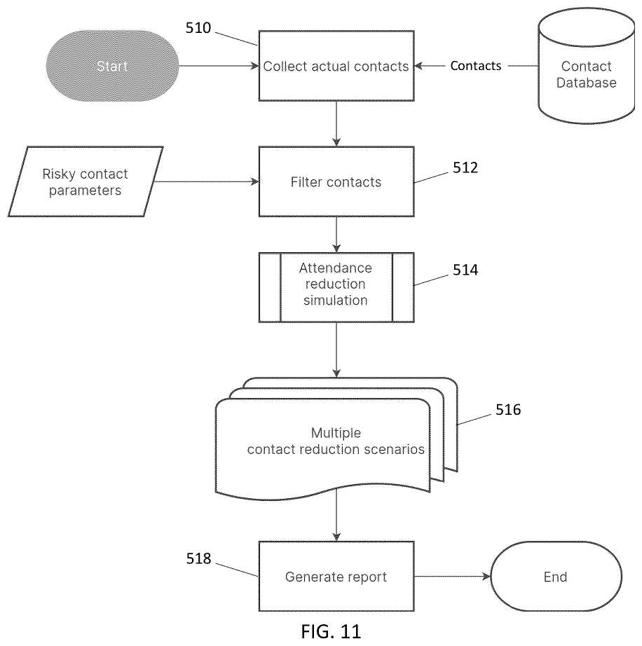

In some embodiments, the system can see all the contacts in conjunction with the attendance of the premises or area being covered. Therefore, a method can be used to simulate lower attendance in order to measure how risky contacts are reduced. This algorithm directly uses the contact data available and statistically computes, using bootstrapping, how reducing attendance reduces contacts and specifically, how it reduces risky contacts. An exemplary embodiment of a method using low attendance to adjust risk assessment is shown in FIG. 11. For example, contacts are collected from a contact database (step 510), the contacts are filtered (step 512) using risk parameters, and an attendance reduction simulation is run (step 514). Multiple contact reduction scenarios are generated (step 516), and a report is created (step 518).

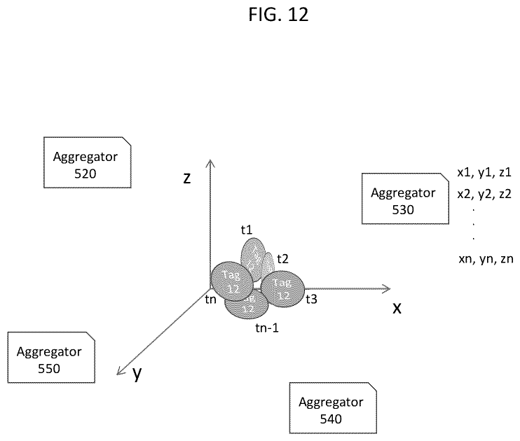

FIG. 12 illustrates an exemplary profiling method. As shown in FIG. 12, a plurality of aggregators 520, 530, 540, 550 are configured to receive coordinates (x1, y1, z1) to (xn, yn, zn) from Tag12 at instants t1 . . . to tn. The coordinates describe a specific point in time for the movement of Tag12 and are recorded by the central computer at every t increment. The coordinates are stored locally in a database and labeled specifically based on the type of movement was executed.

In some embodiments, in order to increase the likelihood of recognizing an event of interest that occurred when the system is deployed, the same movement must be executed thousands of times to ensure a large database of x, y, z values are available. Each movement type is then categorized and stored in the database of the central computer as a mathematical being which can be used instantly when recognizing events which are happening in real life applications. The higher the segmentation (shorter t interval and smaller the value of n), the higher the likelihood that the correct event is recognized.

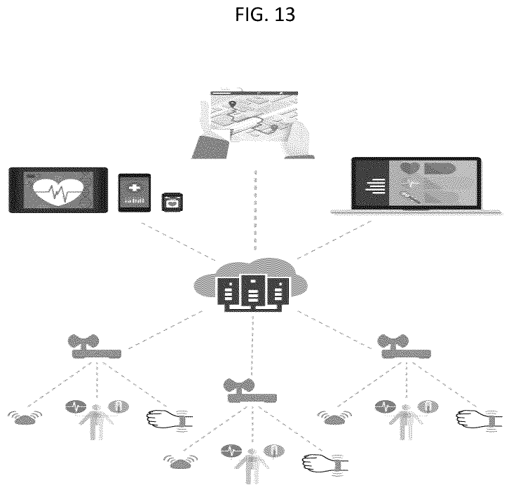

As explained above, the system can also be used for the purpose of locating assets, or devices, anywhere in the specified area, such as a hospital/warehouse area, using an application that utilizes a computing device equipped with wireless capabilities, including but not limited to Bluetooth, Zigbee, NFC, Wi-Fi or any other wireless technologies. FIG. 13 illustrates an embodiment of a system for determining a location and/or status of an asset.

In some embodiments, in order to locate an asset, such as a person, each person is tagged with at least one small tag. The specified area is covered by network, such as a meshed network or any other network that allows for the determination of a general location of the asset in an area (e.g., an entire building, a hospital, the floor of a building). The signal between the asset and the network is strong enough such that the tag placed on the asset can receive commands or other information from the network.

For example, in a hospital implementation, a network of gateways can form an ubiquitous network including a hospital, such that any asset tagged with one of the BLE tags can be “seen” by the network and an approximate location determined by the network of gateways.

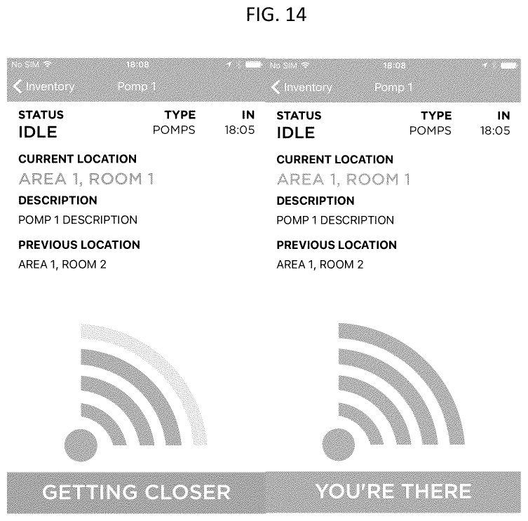

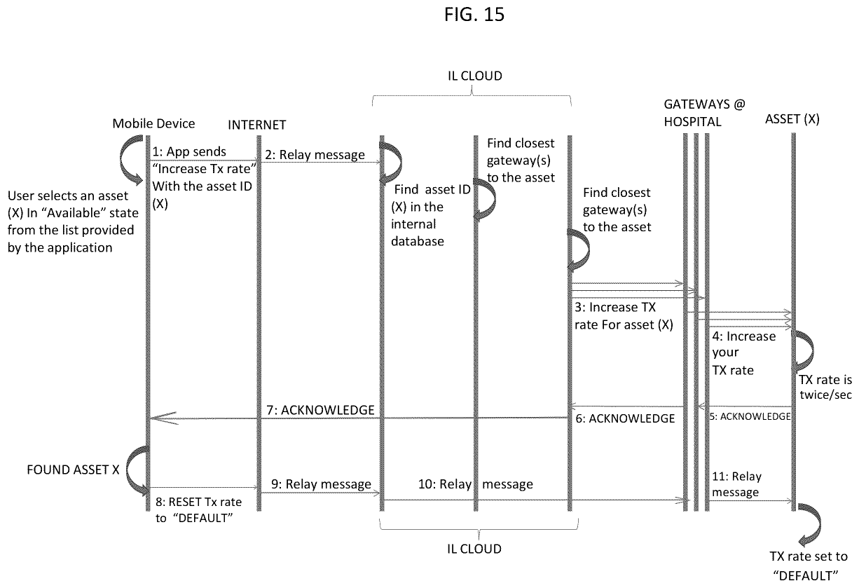

In some embodiments, a mobile application can be used which allows a user, such as a nurse or technician, to use their own device to locate an asset. FIG. 14 illustrates an embodiment of a user interface of a mobile application for locating an asset, and FIG. 15 illustrates an embodiment of a flowchart for determining the location of an asset using a change in a transmission rate of signals from the asset. The mobile application gives the list of assets with their status as described by the algorithms above. When a user selects a specific asset in the list in the mobile application for location, the mobile application (via the mobile device) sends a message to the central computer located in a network, such as the cloud, to increase the transmission rate of that specific BLE associated with the selected asset. For example, the application on Mobile device transmits a message to the central computer to increase the transmission rate to 0.5 times/second. The central computer decodes the message from the mobile application. For example, the central computer determines and finds the identity of the asset/device in its list of registered devices and establishes which one or more gateways are the closest to the current location of the asset. The central computer sends a message to the tag on the asset to increase the transmission rate, for example, to 0.5 times/second. It will be understood that the transmission rate can be increase to any rate that is acceptable for locating the asset.

The one or more gateways receive the message and relay it the tag associated with the desired asset, for example using a radio physical layer radio and a proprietary protocol. Once the asset is located, the user selects the asset from the mobile application running on the mobile device which causes the transmission rate to return to its initial state. The transmission rate can be decreased to the initial rate, allowing the transmission rate from the asset to reset. In some embodiments, one or more safeguards can be put in place such as in the event that the RSSI received by the application on the mobile device is lower than Y dBm (Y depends on the power levels used by the tags to transmit and will be set as an offset to a nominal level and based on the line of sight attenuation of a radio wave at the operating frequency), which can reset the transmission rate to initial rates.

The present disclosure is described with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

These computer program instructions can be provided to a processor of: a general purpose computer to alter its function to a special purpose: a special purpose computer; ASIC: or other programmable digital data processing apparatus, such that the instructions, which execute via the processor of the computer or other programmable data processing apparatus, implement the functions/acts specified in the block diagrams or operational block or blocks, thereby transforming their functionality in accordance with embodiments herein.

For the purposes of this disclosure a computer readable medium (or computer-readable storage medium/media) stores computer data, which data can include computer program code (or computer-executable instructions) that is executable by a computer, in machine readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other physical or material medium which can be used to tangibly store the desired information or data or instructions and which can be accessed by a computer or processor.

For the purposes of this disclosure the term “server” or central computer should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), or other forms of computer or machine readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network. Various types of devices may, for example, be made available to provide an interoperable capability for differing architectures or protocols. As one illustrative example, a router may provide a link between otherwise separate and independent LANs.

A communication link or channel may include, for example, analog telephone lines, such as a twisted wire pair, a coaxial cable, full or fractional digital lines including T1, T2, T3, or T4 type lines, Integrated Services Digital Networks (ISDNs), Digital Subscriber Lines (DSLs), wireless links including satellite links, or other communication links or channels, such as may be known to those skilled in the art. Furthermore, a computing device or other related electronic devices may be remotely coupled to a network, such as via a wired or wireless line or link, for example.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further include a system of terminals, gateways, routers, or the like coupled by wireless radio links, or the like, which may move freely, randomly or organize themselves arbitrarily, such that network topology may change, at times even rapidly.

A wireless network may further employ a plurality of network access technologies, including Wi-Fi, Long Term Evolution (LTE), WLAN, Wireless Router (WR) mesh, or 2nd, 3rd, or 4th generation (2G, 3G, or 4G) cellular technology, or the like. Network access technologies may enable wide area coverage for devices, such as client devices with varying degrees of mobility, for example.

For example, a network may enable RF or wireless type communication via one or more network access technologies, such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, or the like. A wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like. Servers may vary widely in configuration or capabilities, but generally a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

For purposes of this disclosure, a client device, such as, for example, an asset or an aggregator, may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device, a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations. For example, a simple smart phone, phablet or tablet may include a numeric keypad or a display of limited functionality, such as a monochrome liquid crystal display (LCD) for displaying text. In contrast, however, as another example, a web-enabled client device may include a high-resolution screen, one or more physical or virtual keyboards, mass storage, one or more accelerometers, one or more gyroscopes, global positioning system (GPS) or other location-identifying type capability, or a display with a high degree of functionality, such as a touch-sensitive color 2D or 3D display, for example.

A client device may include or may execute a variety of operating systems, including a personal computer operating system, such as a Windows, iOS or Linux, or a mobile operating system, such as iOS, Android, or Windows Mobile, or the like.

A client device may include or may execute a variety of possible applications, such as a client software application enabling communication with other devices, such as communicating one or more messages, such as via email, for example Yahoo!® Mail, short message service (SMS), or multimedia message service (MMS), for example Yahoo! Messenger®, including via a network, such as a social network, including, for example, Tumblr®, Facebook®, LinkedIn®, Twitter®, Flickr®, or Google+®, Instagram™, to provide only a few possible examples. A client device may also include or execute an application to communicate content, such as, for example, textual content, multimedia content, or the like. A client device may also include or execute an application to perform a variety of possible tasks, such as browsing, searching, playing or displaying various forms of content, including locally stored or streamed video, or games. The foregoing is provided to illustrate that claimed subject matter is intended to include a wide range of possible features or capabilities.

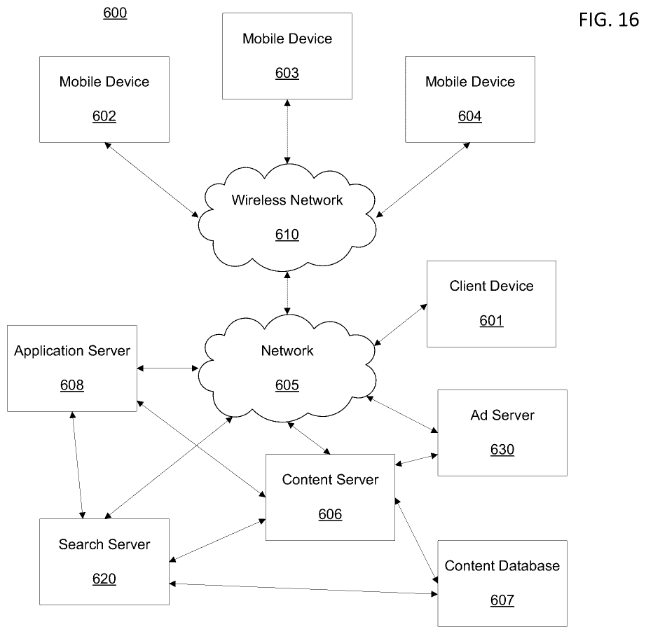

FIG. 16 illustrates an embodiment of a system 600 that is configured to determine a location and/or status of an asset. Not all the components may be required to practice the disclosure, and variations in the arrangement and type of the components may be made without departing from the spirit or scope of the disclosure. As shown, system 600 of FIG. 16 includes a network 605, such as local area networks (“LANs”)/wide area networks (“WANs”), a wireless network 610, mobile devices (client devices) 602-604, and a client device 601. One or more of mobile devices 602-604 and/or client device 601 may be an asset and/or an aggregator. FIG. 16 additionally includes a variety of servers (e.g., central computer), such as a content server 606, an application (or “App”) server 608, a search server 620, and an advertising (“ad”) server 630.

The mobile devices 602-604 can have a variety of forms. In some embodiments, the mobile devices 602-604 can include virtually any portable computing device capable of receiving and sending a message over a network, such as the network 605, the wireless network 610, or the like. The mobile devices 602-604 may also be described generally as client devices that are configured to be portable. Thus, the mobile devices 602-604 may include virtually any portable computing device capable of connecting to another computing device and receiving information. Such devices include, but are not limited to, multi-touch and portable devices such as, cellular telephones, smart phones, display pagers, radio frequency (RF) devices, infrared (IR) devices, Personal Digital Assistants (PDAs), handheld computers, laptop computers, wearable computers, smart watch, tablet computers, phablets, integrated devices combining one or more of the preceding devices, and the like. As such, mobile devices 602-604 can range widely in terms of capabilities and features. For example, a cell phone may have a numeric keypad and a few lines of monochrome LCD display on which only text may be displayed. In another example, a web-enabled mobile device may have a touch sensitive screen, a stylus, and an HD display in which both text and graphics may be displayed.

A web-enabled mobile device can include a browser application that is configured to receive and to send web pages, web-based messages, and the like. The browser application may be configured to receive and display graphics, text, multimedia, and the like, employing virtually any web based language, including a wireless application protocol messages (WAP), and the like. In some embodiments, the browser application is enabled to employ Handheld Device Markup Language (HDML), Wireless Markup Language (WML), WMLScript, JavaScript, Standard Generalized Markup Language (SMGL), HyperText Markup Language (HTML), extensible Markup Language (XML), and the like, to display and send a message.

The mobile devices 602-604 also can include at least one client application that is configured to receive content from another computing device. The client application can include a capability to provide and receive textual content, graphical content, audio content, and the like. The client application may further provide information that identifies itself, including a type, capability, name, and the like. In some embodiments, the mobile devices 602-604 can uniquely identify themselves through any of a variety of mechanisms, including a phone number, Mobile Identification Number (MIN), an electronic serial number (ESN), or other mobile device identifier.

In some embodiments, the mobile devices 602-604 may also communicate with non-mobile client devices, such as the client device 601, or the like. In some embodiments, such communications can include sending and/or receiving messages, searching for, viewing and/or sharing photographs, audio clips, video clips, or any of a variety of other forms of communications. The client device 601 can include virtually any computing device capable of communicating over a network to send and receive information. The set of such devices may include devices that typically connect using a wired or wireless communications medium such as personal computers, multiprocessor systems, microprocessor-based or programmable consumer electronics, network PCs, or the like. Thus, the client device 601 can also have differing capabilities for displaying navigable views of information.

The client device 601 and/or mobile devices 602-604 can be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

The wireless network 610 can be configured to couple the mobile devices 602-604 and its components with the network 605. The wireless network 610 may include any of a variety of wireless sub-networks that may further overlay stand-alone ad-hoc networks, and the like, to provide an infrastructure-oriented connection for the mobile devices 602-604. Such sub-networks may include mesh networks, Wireless LAN (WLAN) networks, cellular networks, and the like.

The network 605 can be configured to couple content server 606, application server 608, or the like, with other computing devices, including but not limited to the client device 601, and through wireless network 610 to the mobile devices 602-604. The network 605 is enabled to employ any form of computer readable media for communicating information from one electronic device to another. Also, the network 605 can include the Internet in addition to local area networks (LANs), wide area networks (WANs), direct connections, such as through a universal serial bus (USB) port, other forms of computer-readable media, or any combination thereof. On an interconnected set of LANs, including those based on differing architectures and protocols, a router acts as a link between LANs, enabling messages to be sent from one to another, and/or other computing devices.

The various networks can employ various protocols to allow for communication over the network. Signal packets communicated via a network, such as a network of participating digital communication networks, may be compatible with or compliant with one or more protocols. Signaling formats or protocols employed may include, for example, TCP/IP, UDP, QUIC (Quick UDP Internet Connection), DECnet, NetBEUI, IPX, APPLETALK™, or the like. Versions of the Internet Protocol (IP) may include IPv4 or IPv6. The Internet refers to a decentralized global network of networks. The Internet includes local area networks (LANs), wide area networks (WANs), wireless networks, or long haul public networks that, for example, allow signal packets to be communicated between LANs. Signal packets may be communicated between nodes of a network, such as, for example, to one or more sites employing a local network address. A signal packet may, for example, be communicated over the Internet from a user site via an access node coupled to the Internet. Likewise, a signal packet may be forwarded via network nodes to a target site coupled to the network via a network access node, for example. A signal packet communicated via the Internet may, for example, be routed via a path of gateways, servers, etc. that may route the signal packet in accordance with a target address and availability of a network path to the target address.

In some embodiments, the system herein can also be utilized within or accessible to an electronic social networking site. A social network refers generally to an electronic network of individuals, such as acquaintances, friends, family, colleagues, or co-workers, that are coupled via a communications network or via a variety of sub-networks. Potentially, additional relationships may subsequently be formed as a result of social interaction via the communications network or sub-networks. In some embodiments, multi-modal communications may occur between members of the social network. Individuals within one or more social networks may interact or communication with other members of a social network via a variety of devices. Multi-modal communication technologies refers to a set of technologies that permit interoperable communication across multiple devices or platforms, such as cell phones, smart phones, tablet computing devices, phablets, personal computers, televisions, set-top boxes, SMS/MMS, email, instant messenger clients, forums, social networking sites, or the like.

In some embodiments, the network 610 and/or the network 605 can comprise a content distribution network(s). A “content delivery network” or “content distribution network” (CDN) generally refers to a distributed content delivery system that comprises a collection of computers or computing devices linked by a network or networks. A CDN may employ software, systems, protocols or techniques to facilitate various services, such as storage, caching, communication of content, or streaming media or applications. A CDN may also enable an entity to operate or manage another's site infrastructure, in whole or in part.

In some embodiment, the content server 606 can include a device that includes a configuration to provide content via a network to another device. The content server 606 can, for example, host a site or service, such as streaming media site/service (e.g., Netflix®), an email platform or social networking site, or a personal user site (such as a blog, vlog, online dating site, and the like). The content server 606 may also host a variety of other sites, including, but not limited to business sites, educational sites, dictionary sites, encyclopedia sites, wikis, financial sites, government sites, and the like. Various devices can operate as the content server 606, including but not limited to personal computers desktop computers, multiprocessor systems, microprocessor-based or programmable consumer electronics, network PCs, servers, and the like.

The content server 606 can further provide a variety of services that include, but are not limited to, streaming and/or downloading media services, search services, email services, photo services, web services, social networking services, news services, third-party services, audio services, video services, instant messaging (IM) services, SMS services, MMS services, FTP services, voice over IP (VOIP) services, or the like. Such services, for example a video application and/or video platform, can be provided via the application server 608, whereby a user is able to utilize such service upon the user being authenticated, verified or identified by the service. Examples of content may include images, text, audio, video, or the like, which may be processed in the form of physical signals, such as electrical signals, for example, or may be stored in memory, as physical states, for example.

An ad server 630 comprises a server that stores online advertisements for presentation to users. “Ad serving” refers to methods used to place online advertisements on websites, in applications, or other places where users are more likely to see them, such as during an online session or during computing platform use, for example. Various monetization techniques or models may be used in connection with sponsored advertising, including advertising associated with user. Such sponsored advertising includes monetization techniques including sponsored search advertising, non-sponsored search advertising, guaranteed and non-guaranteed delivery advertising, ad networks/exchanges, ad targeting, ad serving and ad analytics. Such systems can incorporate near instantaneous auctions of ad placement opportunities during web page creation, (in some cases in less than 500 milliseconds) with higher quality ad placement opportunities resulting in higher revenues per ad. That is, advertisers will pay higher advertising rates when they believe their ads are being placed in or along with highly relevant content that is being presented to users. Reductions in the time needed to quantify a high quality ad placement offers ad platforms competitive advantages. Thus higher speeds and more relevant context detection improve these technological fields.

The servers 606, 608, 620, and 630 can be capable of sending or receiving signals, such as via a wired or wireless network, or may be capable of processing or storing signals, such as in memory as physical memory states. Devices capable of operating as a server may include, as examples, dedicated rack-mounted servers, desktop computers, laptop computers, set top boxes, integrated devices combining various features, such as two or more features of the foregoing devices, or the like. Servers may vary widely in configuration or capabilities, but generally, a server may include one or more central processing units and memory. A server may also include one or more mass storage devices, one or more power supplies, one or more wired or wireless network interfaces, one or more input/output interfaces, or one or more operating systems, such as Windows Server, Mac OS X, Unix, Linux, FreeBSD, or the like.

In some embodiments, users are able to access services provided by the servers 606, 608, 620, and/or 630. This can include in a non-limiting example, authentication servers, search servers, email servers, social networking services servers, SMS servers, IM servers, MMS servers, exchange servers, photo-sharing services servers, and travel services servers, via the network 605 using their various devices 601-604. In some embodiments, applications, such as a streaming video application (e.g., Netflix®, Hulu®, iTunes®, Amazon Prime®, HBO Go®, and the like), blog, photo storage/sharing application or social networking application (e.g., Flickr®, Tumblr®, and the like), can be hosted by the application server 608 (or the content server 606, the search server 620, and the like). Thus, the application server 608 can store various types of applications and application related information including application data and user profile information (e.g., identifying and behavioral information associated with a user). It should also be understood that the content server 606 can also store various types of data related to the content and services provided by the content server 606 in an associated content database 607.

Moreover, although FIG. 16 illustrates the servers 606, 608, 620, and 630 as single computing devices, respectively, in some embodiments, one or more functions of the servers 606, 608, 620, and/or 630 can be distributed across one or more distinct computing devices. Moreover, in some embodiments, the servers 606, 608, 620 and/or 630 may be integrated into a single computing device.

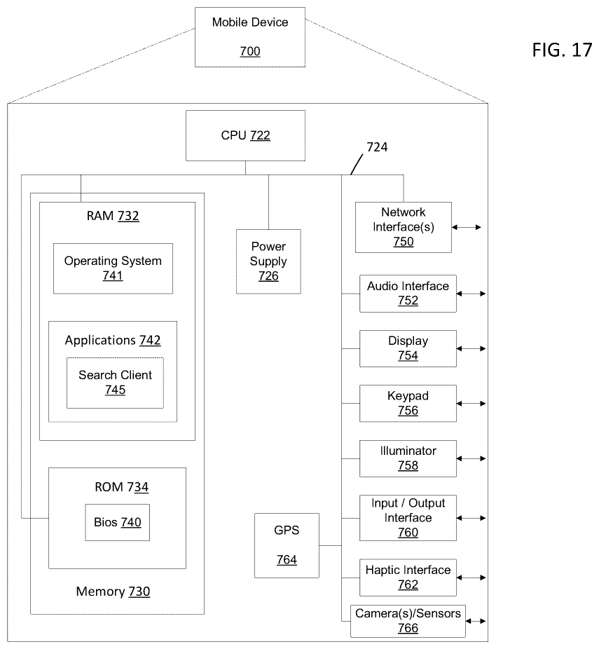

FIG. 17 illustrates an exemplary embodiment of a client device (e.g., an asset). A client device 700 can include many more or less components than those shown in FIG. 17. The client device 700 can represent, for example, client devices discussed above in relation to FIG. 16.

As shown in FIG. 17, the client device 700 includes a processing unit (CPU) 722 in communication with a mass memory 730 via a bus 724. The client device 700 also includes a power supply 726, one or more network interfaces 750, an audio interface 752, a display 754, a keypad 756, an illuminator 758, an input/output interface 760, a haptic interface 762, an optional global positioning systems (GPS) receiver 764 and a camera(s) or other optical, thermal or electromagnetic sensors 766. The client device 700 can include one camera/sensor 766, or a plurality of cameras/sensors 766, as understood by those of skill in the art. The positioning of the camera(s)/sensor(s) 766 on the client device 700 can change per client device 700 model, per client device 700 capabilities, and the like, or some combination thereof.

The power supply 726 provides power to the client device 700. A rechargeable or non-rechargeable battery may be used to provide power. The power may also be provided by an external power source, such as an AC adapter or a powered docking cradle that supplements and/or recharges a battery.

The client device 700 may optionally communicate with a base station (not shown), or directly with another computing device. The network interface 750 includes circuitry for coupling the client device 700 to one or more networks, and is constructed for use with one or more communication protocols and technologies as discussed above. The network interface 750 is sometimes known as a transceiver, transceiving device, or network interface card (NIC).

The audio interface 752 can be arranged to produce and receive audio signals such as the sound of a human voice. For example, the audio interface 752 can be coupled to a speaker and microphone (not shown) to enable telecommunication with others and/or generate an audio acknowledgement for some action. The display 754 can be a liquid crystal display (LCD), gas plasma, light emitting diode (LED), or any other type of display used with a computing device. The display 754 can also include a touch sensitive screen arranged to receive input from an object such as a stylus or a digit from a human hand.

The keypad 756 can comprise any input device arranged to receive input from a user. For example, the keypad 756 can include a push button numeric dial, or a key board. The keypad 756 can also include command buttons that are associated with selecting and sending images. Illuminator 758 may provide a status indication and/or provide light. Illuminator 758 may remain active for specific periods of time or in response to events. For example, when the illuminator 758 is active, it can backlight the buttons on the keypad 756 and stay on while the client device is powered. Also, the illuminator 758 can backlight these buttons in various patterns when particular actions are performed, such as dialing another client device. Illuminator 758 may also cause light sources positioned within a transparent or translucent case of the client device to illuminate in response to actions.

The client device 700 also comprises input/output interface 760 for communicating with external devices, such as a headset, or other input or output devices not shown in FIG. 17. The input/output interface 760 can utilize one or more communication technologies, such as USB, infrared, Bluetooth™, or the like. Haptic interface 762 is arranged to provide tactile feedback to a user of the client device. For example, the haptic interface 762 can be employed to vibrate the client device 700 in a particular way when the client device 700 receives a communication from another user.

Optional GPS transceiver 764 can determine the physical coordinates of The client device 700 on the surface of the Earth, which typically outputs a location as latitude and longitude values. GPS transceiver 764 can also employ other geo-positioning mechanisms, including, but not limited to, triangulation, assisted GPS (AGPS), E-OTD, CI, SAI, ETA, BSS or the like, to further determine the physical location of Client device 700 on the surface of the Earth. It is understood that under different conditions, GPS transceiver 764 can determine a physical location within millimeters for the client device 700; and in other cases, the determined physical location may be less precise, such as within a meter or significantly greater distances. In some embodiments, however, the client device can through other components, provide other information that may be employed to determine a physical location of the device, including for example, a MAC address, Internet Protocol (IP) address, or the like.

Mass memory 730 includes a RAM 732, a ROM 734, and other storage means. The mass memory 730 illustrates another example of computer storage media for storage of information such as computer readable instructions, data structures, program modules or other data. The mass memory 730) stores a basic input/output system (“BIOS”) 740 for controlling low-level operation of the client device 700. The mass memory 730 also stores an operating system 741 for controlling the operation of the client device 700. It will be appreciated that this component may include a general purpose operating system such as a version of UNIX, or LINUX™, or a specialized client communication operating system such as Windows Client™, or the Symbian® operating system. The operating system 741 can include, or interface with a Java virtual machine module that enables control of hardware components and/or operating system operations via Java application programs.

The memory 730 can further include one or more data stores, which can be utilized by the client device 700 to store, among other things, applications 742 and/or other data. For example, data stores may be employed to store information that describes various capabilities of the client device 700. The information may then be provided to another device based on any of a variety of events, including being sent as part of a header during a communication, sent upon request, or the like. At least a portion of the capability information may also be stored on a disk drive or other storage medium (not shown) within the client device 700.

The applications 742 can include computer executable instructions which, when executed by the client device 700, transmit, receive, and/or otherwise process audio, video, images, and enable telecommunication with a server and/or another user of another client device. Other examples of application programs or “apps” in some embodiments include browsers, calendars, contact managers, task managers, transcoders, photo management, database programs, word processing programs, security applications, spreadsheet programs, games, search programs, and so forth. The applications 742 can also include a search client 745 that is configured to send, to receive, and/or to otherwise process a search query and/or search result using any known or to be known communication protocols. Although a single search client 745 is illustrated it should be clear that multiple search clients may be employed. For example, one search client may be configured to enter a search query message, where another search client manages search results, and yet another search client is configured to manage serving advertisements, IMs, emails, and other types of known messages, or the like.

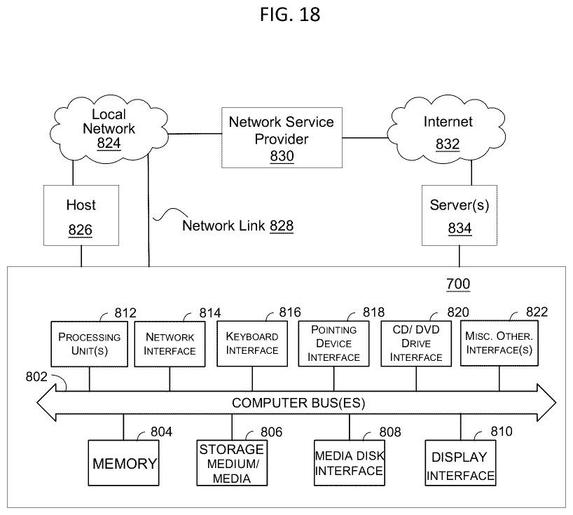

As shown in FIG. 18, in some embodiments an internal architecture 800 of a computing device(s) (e.g., an asset), computing system, computing platform and the like can include one or more processing units, processors, or processing cores, (also referred to herein as CPUs) 812, which interface with at least one computer bus 802. Also interfacing with the computer bus 802 are, for example, a computer-readable medium, or media, 806, a network interface 814, a memory 804, e.g., random access memory (RAM), run-time transient memory, read only memory (ROM), a media disk drive interface 820 as an interface for a drive that can read and/or write to media including removable media such as floppy, CD-ROM, DVD, media, a display interface 710 as interface for a monitor or other display device, a keyboard interface 816 as interface for a keyboard, a pointing device interface 818 as an interface for a mouse or other pointing device, and miscellaneous other interfaces not shown individually, such as parallel and serial port interfaces and a universal serial bus (USB) interface.

The memory 804 interfaces with the computer bus 802 so as to provide information stored in memory 804 to CPU 812 during execution of software programs such as an operating system, application programs, device drivers, and software modules that comprise program code, and/or computer executable process steps, incorporating functionality described herein, e.g., one or more of process flows described herein. CPU 812 first loads computer executable process steps from storage, e.g., the memory 804, computer readable storage medium/media 806, removable media drive, and/or other storage device. CPU 812 can then execute the stored process steps in order to execute the loaded computer-executable process steps. Stored data, e.g., data stored by a storage device, can be accessed by CPU 812 during the execution of computer-executable process steps.

Persistent storage, e.g., medium/media 806, can be used to store an operating system and one or more application programs. Persistent storage can also be used to store device drivers, such as one or more of a digital camera driver, monitor driver, printer driver, scanner driver, or other device drivers, web pages, content files, playlists and other files. Persistent storage can further include program modules and data files used to implement one or more embodiments of the present disclosure.

The network link 828 can provide information communication using transmission media through one or more networks to other devices that use or process the information. For example, the network link 828 can provide a connection through the local network 824 to a host computer 826 or to equipment operated by a Network or Internet Service Provider (ISP) 830. ISP equipment in turn provides data communication services through the public, worldwide packet-switching communication network of networks now commonly referred to as the Internet 832.

A computer called a server host 834 connected to the Internet 832 hosts a process that provides a service in response to information received over the Internet 832. For example, the server host 834 hosts a process that provides information representing video data for presentation at the display 810. Various components of the system 800 can be deployed in various configurations within other computer systems, e.g., host and server.

In some embodiments, the computer system 800 can be used for implementing some or all of the techniques described herein. In some embodiments, those techniques are performed by computer system 800 in response to the processing unit 812 executing one or more sequences of one or more processor instructions contained in the memory 804. Such instructions, also called computer instructions, software and program code, can be read into the memory 804 from another computer-readable medium 806 such as storage device or network link. Execution of the sequences of instructions contained in the memory 804 causes the processing unit 812 to perform one or more of the method steps described herein. In some embodiments, hardware, such as ASIC, can be used in place of or in combination with software. Thus, embodiments of the present disclosure are not limited to any specific combination of hardware and software, unless otherwise explicitly stated herein.

The signals transmitted over network link and other networks through communications interface, carry information to and from the computer system 800. The computer system 800 can send and receive information, including program code, through the networks, among others, through network link and communications interface. In an example using the Internet, a server host transmits program code for a particular application, requested by a message sent from computer, through Internet, ISP equipment, local network and communications interface. The received code may be executed by the processor 802 as it is received, or may be stored in the memory 804 or in storage device or other non-volatile storage for later execution, or both.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers or be loaded and executed by one or more servers. One or more modules may be grouped into an engine or an application.

Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A contact tracing system, comprising:
a plurality of aggregators configured to communicate in a meshed network to create a geo-fence within a designated area, each of the plurality of aggregators comprising a processor and a transceiver;
one or more sensors, each of the one or more sensors associated with an asset, the one or more sensors configured to communicate, with the plurality of aggregators, information relating to a location of the asset based on a relative location of the asset to each of the plurality of aggregators; and
a central computer in communication with the plurality of aggregators and configured to receive information from at least one of the plurality of aggregators relating to a contact tracing event within the geo-fence within the designated area, the central computer configured to
receive, from at least one of the plurality of aggregators, a plurality of parameters relating to an asset in the designated space; and
determining a contact status of the asset based on the plurality of parameters, the plurality of parameters including a duration of time the at least one asset remained in the designated area and a distance between the at least one asset and one or more additional assets in the designated area.

2. The system of claim 1, wherein the central computer is configured to calculate a proximity score using one or more signals from the sensors as captured by each aggregator, the one or more signal being transformed into a single numerical vector, the proximity score being a similarity between a first numerical vector related to the at least one asset and a second numerical vector related to one or the one or more additional assets.

3. The system of claim 2, wherein the central computer is configured to compare the proximity score with a stored proximity threshold value to determine if the asset and the additional asset are in close proximity indicative of spreading disease.

4. The system of claim 1, wherein the central computer is configured to calculate a contact duration score using timestamps of all the signals received by the aggregators in the designated area.

5. The system of claim 4, wherein the central computer is configured to compare the contact duration score with a stored duration threshold value to determine if the asset and the additional asset are in contact long enough to be indicative of spreading disease.

6. The system of claim 1, wherein the central computer is configured to create a contact database of contacts between the asset and the one or more additional assets for automatic contact tracing.

7. A method of contact tracing, comprising:
transmitting a plurality of parameters from one or more of a plurality of aggregators relating to an asset to a central computer, the asset including one or more tags positioned thereon for communicating with the plurality of aggregators in a designated area;
determining a localization, by the central computer, of the asset in the designated area based on the plurality of parameters,
determining a contact between the asset and one or more additional assets based on the determined localization of the asset and the one or more additional assets;
determining a contact status of the asset based on the plurality of parameters using an algorithm on the central computer, the plurality of parameters including a duration of time the asset remains in the designated area and a distance between the asset and one or more additional assets in the designated area; and
determining a close contact between the asset and the one or more additional assets, a close contact defined as a contact between the asset and the additional asset indicative of spreading disease.

8. The method of claim 7, further comprising calculating a proximity score using one or more signals from the tags as captured by each aggregator, the one or more signals being transformed into a single numerical vector, the proximity score being a similarity between a first numerical vector related to the asset and a second numerical vector related to one or the one or more additional assets.

9. The method of claim 8, further comprising comparing the proximity score with a stored proximity threshold value to determine if the asset and the additional asset are in close proximity indicative of spreading disease.

10. The method of claim 7, further comprising calculating a contact duration score using timestamps of all the signals received by the aggregators in the designated area.

11. The method of claim 10, further comprising comparing the contact duration score with a stored duration threshold value to determine if the asset and the additional asset are in contact long enough to be indicative of spreading disease.

12. The method of claim 7, further comprising generating an alert if the contact is determined to be a close contact indicative of spreading disease.

13. The method of claim 7, wherein the central computer creates a contact database of the close contacts between the asset and the one or more additional assets for automatic contact tracing.

14. The method of claim 7, further comprising creating a geo-fence within the designated area defining the designated area using a plurality of aggregators, each aggregator capable of communicating with neighboring aggregators and with the central computer over a network.

15. A method of contact tracing, comprising:
transmitting a plurality of parameters from one or more of a plurality of aggregators relating to one or more assets to a central computer, each of the one or more assets including one or more tags positioned thereon for communicating with the plurality of aggregators in a designated area, the one or more assets including at least a first asset and a second asset;
determining, by a central computer, a proximity score related to a proximity between the first asset and the second asset based on the plurality of parameters from the plurality of parameters;
determining, by a central computer, a duration score related to a duration of time the first asset and the second asset are in the designated area based on the plurality of parameters from the plurality of parameters; and
determining a contact status of the first asset and the second asset based on the proximity score and the duration score using an algorithm on the central computer; the algorithm comparing the proximity score to a threshold proximity value stored on the central computer and comparing the duration score to a threshold duration value stored on the central computer.

16. The method of claim 15, wherein the contact status is determined to be a close contact when the proximity score exceeds the threshold proximity value and the duration score exceeds the threshold duration value, indicating a contact that is indicative of spreading disease.

17. The method of claim 15, wherein the central computer creates a contact database of the close contacts between the asset and the one or more additional assets for automatic contact tracing.

18. The method of claim 15, further comprising creating a geo-fence within the designated area defining the designated area using a plurality of aggregators, each aggregator capable of communicating with neighboring aggregators and with the central computer over a network.

19. The method of claim 15, further comprising calculating a proximity score using one or more signals from the tags as captured by each aggregator, the one or more signal being transformed into a single numerical vector, the proximity score being a similarity between a first numerical vector related to the at least one asset and a second numerical vector related to one or the one or more additional assets.

20. The method of claim 15, further comprising calculating a contact duration score using timestamps of all the signals received by the aggregators in the designated area.
